(19) United States
(12) Patent Application Publication
(10) Pub. No.: US 2023/0053843 A1
(43) Pub. Date: Feb. 23, 2023

(54) COMPOSITION FOR BRIGHTENING OR WHITENING KERATIN MATERIALS

(71) Applicant: L'OREAL, Paris (FR)

(72) Inventors: Chi MA, Shanghai (CN); Xiuxia WANG, Shanghai (CN)

(73) Assignee: L'OREAL, Paris (FR)

(21) Appl. No.: 17/789,870

(22) PCT Filed: Dec. 30, 2019
(86) PCT No.: PCT/CN2019/129826

§ 371 Date: Jun. 29, 2022

(51) Int. Cl. A61K 8/06; A61K 8/37; A61K 8/81; A61K 8/55; A61Q 19/02; A61K 8/19; A61K 8/29; A61K 8/25

(52) U.S. Cl. CPC A61K 8/062; A61K 8/19; A61K 8/25; A61K 8/29; A61K 8/55; A61K 8/375; A61K 8/8152; A61Q 19/02

(57) ABSTRACT

A composition for brightening or whitening keratin materials in the form of an emulsion, comprises an oily phase dispersed in an aqueous phase, and comprises: i)from 2.0 wt. % to 10.0 wt% of at least one white particles, relative the total weight of the composition; ii) at least one anionic terpolymer as a thickener; and iii) an emulsifier system including: a)at least one alkyl phosphate salt; b)at least one polyoxyethylene oxide C12-C24 fatty acid ester; and c)at least one glyceryl C12-C24 fatty acid ester. A cosmetic process for brightening or whitening keratin materials, in particular human skin, comprises applying the composition as defined above to the keratin materials.

### TECHNICAL FIELD

The present invention relates to a cosmetic composition. In particular, the present invention relates to a composition for brightening or whitening keratin materials, in particular human skin.

### BACKGROUND ART

Human skin colour depends on various factors, and in particular on the seasons of the year, on race and on gender; it is mainly determined by the nature and the concentration of melanin produced by melanocytes. Melanocytes are specialized cells which synthesize melanin by means of specific organelles, melanosomes. In addition, at various times in their life, certain individuals experience the appearance of dark and/or coloured spots on the skin and more especially on the hands, which give the skin heterogeneity.

For various reasons associated in particular with greater comfort of use (softness, emollience and the like), current compositions for caring for and/or making up keratin materials, in particular the skin, are usually in the form of an emulsion of the oil-in-water (O/W) type consisting of an aqueous dispersing continuous phase and an oily dispersed discontinuous phase, or of an emulsion of the water-in-oil (W/O) type consisting of an oily dispersing continuous phase and an aqueous dispersed discontinuous phase.

O/W emulsions are the ones most sought in the cosmetics field, since they comprise an aqueous phase as the external phase, which gives them, when applied to the skin, a fresher, less greasy and lighter feel than W/O emulsions.

Efforts have also been made to introduce whitening pigments into the oil-in-water emulsions. Unfortunately, such pigments generally negatively impact the stability of the emulsions, thereby somewhat complicating the manufacture of the emulsion.

In addition, the conventional oil-in-water emulsions are not totally satisfying, in particular in terms of long-term brightening or whitening of the skin, since the content of the brightening or whitening active ingredient is low.

Therefore, there exists a need for formulating a composition comprising a higher content of brightening or whitening active ingredient and being stable, meanwhile having a suitable viscosity so that it is convenient for the consumers to apply them on the keratin materials.

### SUMMARY OF THE INVENTION

The inventors have found that such a need can be achieved by the present invention.

Thus, according to a first aspect, the present invention provides a composition for brightening or whitening keratin materials in the form of an emulsion, comprising an oily phase dispersed in an aqueous phase, and comprising:
i) from 2.0 wt.% to 10.0 wt% of at least one white particles, relative the total weight of the composition;
ii) at least one anionic terpolymer as a thickener; and
iii)an emulsifier system including:
   a) at least one alkyl phosphate salt;
   b) at least one polyoxyethylene oxide C12-C24 fatty acid ester; and
   c) at least one glyceryl C12-C24 fatty acid ester.

The composition according to the present invention is advantageous in several respects.

Firstly, the composition according to the present invention has a brightening or whitening effect to keratin materials, in particular human skin.

Secondly, the composition of the present invention has a suitable viscosity so that it is convenient for the consumers to apply them on the keratin materials.

In addition, the composition of the present invention is stable.

According to a second aspect, the present invention provides a cosmetic process for brightening or whitening keratin materials, in particular human skin, comprising applying the composition according to the present invention on the keratin materials.

Other advantages of the present invention will emerge more clearly on reading the description and the examples that follow.

### DETAILED DESCRIPTION OF THE INVENTION

According to the first aspect, the present invention provides a composition for brightening or whitening keratin materials in the form of an emulsion, comprising an oily phase dispersed in an aqueous phase, and comprising:
i) from 2.0 wt.% to 10.0 wt. % of at least one white particles, relative the total weight of the composition;
ii) at least one anionic terpolymer as a thickener; and
iii) an emulsifier system including:
   a) at least one alkyl phosphate salt;
   b) at least one polyoxyethylene oxide C12-C24 fatty acid ester; and
   c) at least one glyceryl C12-C24 fatty acid ester.

In that which follows and unless otherwise indicated, the limits of a range of values are included within this range, in particular in the expressions “between... and” and “from ... to ...”.

For the purposes of the present invention, the term “keratin materials” are intended to cover human skin, mucous membranes such as the lips. Human skin, in particular face and neck, are most particularly considered according to the present invention.

Moreover, the expression “at least one” used in the present description is equivalent to the expression “one or more”.

Throughout the instant application, the term “comprising” is to be interpreted as encompassing all specifically mentioned features as well optional, additional, unspecified ones.

As used herein, the use of the term “comprising” also discloses the embodiment wherein no features other than the specifically mentioned features are present (i.e. “consisting of”).

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

Unless otherwise specified, all numerical values expressing amount of ingredients and the like which are used in the description and claims are to be understood as being modified by the term “about”. Accordingly, unless indicated to the contrary, the numerical values and parameters described herein are approximate values which are capable of being changed according to the desired purpose as required.

### White Particles

The term “white” is intended to mean particles having the colour white and its derivatives (off-white, alabaster white, etc.), as opposed to primary colours and derivatives.

In the present application, white particles cover particles being white, off-white, or alabaster white from inside to outside, and also cover particles being white, off-white, or alabaster white inside and other color outside, or being white, off-white, or alabaster white outside and other color inside, on the condition that the white part is at least 90% by volume based on the whole volume of the particles.

In some embodiment, the white particles used are inorganic particles.

In particular, the white particles have a lightness value L* close to 100 in the CIELab76 system.

Preferably, the white particles is characterized by 0.3 µm<D50<40 µm.

The volume average size (D50) is a parameter for particle size distribution, referring to the maximum particle diameter below which 50% of the sample volume exists (see in “A Basic Guide To Particle Characterization”, page 10, published by Malvern Instruments Limited in 2012).

The volume average size (D50) of the particles may be measured by static light scattering using a commercial granulometer such as the MasterSizer 3000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H.C., “Light Scattering by Small Particles,” Chapters 9 and 10, Wiley, New York, 1957.

The white particles according to the present invention have a continuous effect, which is controlled by the resolution capacity of the eye. Since said capacity is around 40 µm, particles having a volume average size (D50) of less than 40 µm, preferably less than 25 µm and even better still less than or equal to 6 µm are therefore considered. However, it is ensured that particles of which the D50 is greater than 0.3 µm and preferentially 0.5 µm are taken, so as not to have too great a loss of opacity, which would be extremely detrimental to the desired effect.

Thus, the composition of the present invention preferably comprises at least white particles having a volume size (D50) of less than 40 µm, preferably between 0.3 and 25 µm and better still between 0.3 and 6 µm, or even between 0.5 and 3 µm.

These particles may be made of one material or composites. For example, these particles may be selected from boron nitride, titanium dioxide, silica, pearl, mica, synthetic mica, mica based pearl pigment particles, composite particles comprising titanium dioxide and a substrate selected from alumina, silica, barium sulfate, glass, mica and synthetic mica, or mixtures thereof.

As examples of composite particles, mention may, for example, be made of platelets of mica covered with TiO₂.

By way of example of particles, mention may be made of boron nitride particles, such as PUHP1030L from Saint Gobain Ceramics and UHP-1010 from Carborundum.

According to a preferred embodiment, the particles are selected from boron nitride, titanium dioxide, mica and a mixture thereof.

In some embodiments, composite particles comprising titanium dioxide and a mica substrate are used.

In some embodiments, a combination of boron nitride particles and composite particles comprising titanium dioxide and a mica substrate is used.

Advantageously, the white particles are present in the composition according to the present invention in an amount ranging from 2.5 wt.% to 8.0 wt.%, preferably from 3 wt.% to 6 wt.%, relative to the total weight of the composition.

### Anionic Terpolymer

According to the first aspect, the composition according to the present invention comprises at least one anionic terpolymer as a thickener.

The anionic terpolymer may be a linear or branched and/or crosslinked terpolymer, of at least one monomer (1) bearing an acid function being in free or partially or totally salified form with a nonionic monomer (2) chosen from N,N-dimethylacrylamide and 2-hydroxyethyl acrylate and at least one polyoxyethylenated alkyl acrylate monomer (3) of formula (I) below:

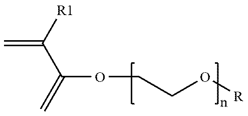

in which:
R1 represents a hydrogen atom,
R represents a linear or branched C8-C16 alkyl radical, and
n represents a number ranging from 1 to 10.

The term “branched polymer” denotes a non-linear polymer which bears pendent chains so as to obtain, when this polymer is dissolved in water, a high degree of entanglement leading to very high viscosities, at a low speed gradient.

The term “crosslinked polymer” denotes a non-linear polymer which is in the form of a three-dimensional network that is insoluble in water but swellable in water, leading to the production of a chemical gel.

The acid function of the monomer (1) may be a sulfonic acid or phosphonic acid function, said functions being in free or partially or totally salified form.

The monomer (1) may be chosen from styrenesulfonic acid, ethylsulfonic acid and 2-methyl-2-[(1-oxo-2-propenyl]amino]-1-propanesulfonic acid (also known as acryloyldimethyl taurate), in free or partially or totally salified form. It may be present in the anionic terpolymer in molar proportions ranging from 5 mol % to 95 mol %, such as from 10 mol % to 90 mol %. The monomer (1) may be 2-methyl-2-[(1-oxo-2-propenyl)amino]-1-propanesulfonic acid in free or partially or totally salified form.

The acid function in partially or totally salified form will preferably be an alkali metal salt such as a sodium or potassium salt, an ammonium salt, an amino alcohol salt such as a monoethanolamine salt, or an amino acid salt such as a lysine salt.

The monomer (2) may be present in the anionic terpolymer in molar proportions ranging from 4.9 mol % to 90 mol %, such as from 9.5 mol % to 85 mol %, or from 19.5 mol % to 75 mol %.

In formula (I), examples of linear C8-C16 alkyl radicals that may be mentioned include octyl, decyl, undecyl, tridecyl, tetradecyl, pentadecyl and hexadecyl.

In formula (I), examples of branched C8-C16 alkyl radicals that may be mentioned include 2-ethylhexyl, 2-propylheptyl, 2-butyloctyl, 2-pentylnonyl, 2-hexyldecyl, 4-methylpentyl, 5-methylhexyl, 6-methylheptyl, 15-methylpentadecyl, 16-methylheptadecyl and 2-hexyloctyl.

According to one exemplary embodiment, in formula (I), R is chosen from a C12-C16 alkyl radical.

According to one exemplary embodiment, in formula (I), n ranges from 3 to 5.

Tetraethoxylated lauryl acrylate may be chosen as monomer of formula (I).

The monomer (3) of formula (I) may be present in the anionic terpolymer in molar proportions ranging from 0.1 mol % to 10 mol %, for example from 0.5 mol % to 5 mol %.

According to one exemplary embodiment, the anionic terpolymer is crosslinked and/or branched with a diethylenic or polyethylenic compound in the proportion expressed relative to the total amount of monomers used, ranging from 0.005 mol % to 1 mol %, such as from 0.01 mol % to 0.5 mol %, or from 0.01 mol % to 0.25 mol %.

The crosslinking agent and/or branching agent may be chosen from ethylene glycol dimethacrylate, diallyloxyacetic acid or a salt thereof, such as sodium diallyloxyacetate, tetraallyloxyethane, ethylene glycol diacrylate, diallylurea, triallylamine, trimethylolpropane triacrylate and methylenebis(acrylamide), or mixtures thereof.

The anionic terpolymer may contain additives such as complexing agents, transfer agents, or chain-limiting agents.

In various exemplary embodiments, anionic terpolymers of 2-methyl-2-[(1-oxo-2-propenyl)amino]-1-propanesulfonic acid partially or totally salified in the form of the ammonium salt, N,N-dimethylacrylamide and tetraethoxylated lauryl acrylate crosslinked with trimethylolpropane triacrylate, of INCI name Polyacrylate Crosspolymer-6, such as the product sold under the trade name Sepimax Zen® by the company SEPPIC, may be used.

The presence of anionic terpolymer provides the composition according to the present invention a suitable viscosity.

Advantageously, the anionic terpolymer is present in the composition according to the present invention in an amount ranging from 0.1 wt.% to 5 wt.%, preferably from 0.2 wt.% to 2 wt.%, and more preferably from 0.3 wt.% to 1 wt.%, relative to the total weight of the composition.

### Emulsifier System

According to the first aspect, the composition according to the present invention comprises a emulsifier system including alkyl phosphate salt, polyoxyethylene oxide C12-C24 fatty acid ester, and glyceryl C12-C24 fatty acid ester.

### Alkyl Phosphate Salts

According to the first aspect, the composition according to the present invention comprises at least one alkyl phosphate salt.

In some embodiments, the alkyl phosphate salt is a dialkyl phosphate salt or a monoalkyl phosphate salt. Preferably, the alkyl phosphate salt is a monoalkylphosphate salt.

As used herein, the term “monoalkyl” means that the phosphate unit is linked to only one alkyl chain.

As used herein, the term “dialkyl” means that the phosphate unit is linked to two alkyl chains.

In one embodiment, the monoalkyl phosphate salt possesses the following formula (II):

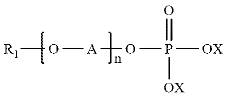

wherein:
R₁ represents a linear or branched, alkyl or alkylene comprising from 4 to 24 carbon atoms, preferably from 12 to 22 carbon atoms, more preferably from 16 to 18 carbon atoms;
A represents an alkylene group comprising from 2 to 10 carbon atoms;
n represents an integer ranging from 0 to 20, preferably n is 0;
X represents alkali metals, such as potassium, lithium and sodium.

In some embodiments, the dialkyl phosphate salt is a dicetyl phosphate salt, such as a potassium dicetyl phosphate.

In some embodiments, the monoalkyl phosphate salt is a potassium cetyl phosphate, for example commercialized under the trade names Amphisol® K (Roche), Amphisol® A (Roche), Arlatone® MAP (Uniqema), Crodafos® MCA (Croda).

Advantageously, the alkyl phosphate salt is present in the composition according to the present invention in an amount ranging from 0.1 wt.% to 10 wt.%, preferably from 0.3 wt.% to 5 wt.%, and more preferably from 0.5 wt.% to 2 wt.%, based on the total weight of the composition.

### Polyoxyethylene Oxide C12-C24 Fatty Acid Ester

The polyoxyethylene oxide portion of the C12-C24 fatty acid ester is a polyethylene glycol (PEG) generally having 50 to 150, preferably 75 to 120, more preferably 80 to 100 moles or units of PEG, e.g., PEG 75 and PEG 100.

The C12-C24 fatty acid can be unsaturated or saturated acid, for example, stearic acid, oleic acid, palmitic acid.

As examples of polyoxyethylene oxide C12-C24 fatty acid ester, mention can be made to PEG 75 stearate, PEG 100 stearate, PEG 75 oleate, PEG 100 oleate, PEG 75 palmitate and PEG 100 palmite.

Preferably, the polyoxyethylene oxide C12-C24 fatty acid ester used is PEG 100 stearate.

Advantageously, the polyoxyethylene oxide C12-C24 fatty acid ester is present in the composition according to the present invention in an amount ranging from 0.1 wt.% to 10 wt.%, preferably from 0.3 wt.% to 5 wt.%, and more preferably from 0.5 wt.% to 2 wt.%, based on the total weight of the composition.

### Glyceryl C12-C24 Fatty Acid Ester

The C12-C24 fatty acid for the glyceryl C12-C24 fatty acid can be unsaturated or saturated acid, for example, stearic acid, oleic acid, palmitic acid.

As examples of glyceryl C12-C24 fatty acid ester, mention can be made to glyceryl laurate, glyceryl oleate, glyceryl stearate and glyceryl palmitate.

As commercialized products, mention can be made to glyceryl stearate such as the product sold under the name Tegin M® by the company Evonik Goldschmidt, glyceryl laurate such as the product sold under the name Imwitor 312® by the company Hiils.

Preferably, the glyceryl C12-C24 fatty acid ester used is glyceryl stearate.

Advantageously, the glyceryl C12-C24 fatty acid ester is present in the composition according to the present invention in an amount ranging from 0.1 wt.% to 10 wt.%, preferably from 0.3 wt.% to 5 wt.%, and more preferably from 0.5 wt.% to 2 wt.%, based on the total weight of the composition.

In some embodiments, the weight ratio R of alkyl phosphate salt, polyoxyethylene oxide C12-C24 fatty acid ester and glyceryl C12-C24 fatty acid ester as defined below is 1 : 0.5-2 :0.5-2, preferably 1: 0.8-1.5:0.8-1.5.

R = alkyl phosphate salt/polyoxyethylene oxide C12-C24 fatty acid ester/ glyceryl C12-C24 fatty acid ester.

In some embodiments, glyceryl stearate and PEG-100 Stearate (for example such as the product sold under name ARLACEL™ 165 from CRODA) are used in the composition according to the present invention.

In some embodiments, potassium cetyl phosphate, glyceryl stearate and PEG-100 stearate are used in the composition according to the present invention.

### Aqueous Phase

The composition of the present invention comprises at least one continuous aqueous phase.

The aqueous phase of the composition according to the present invention comprises water and optionally one or more water-miscible or at least partially water-miscible compounds, for instance C₂ to C₈ lower polyols or monoalcohols, such as ethanol and isopropanol.

The term “polyol” should be understood as meaning any organic molecule comprising at least two free hydroxyl groups. Examples of polyols that may be mentioned include glycols, for instance propylene glycol, butylene glycol, pentylene glycol, and isoprene glycol, caprylyl glycol, glycerol (i.e. glycerin) and polyethylene glycols.

The aqueous phase may also comprise any common water-soluble or water-dispersible additive as mentioned below.

The aqueous phase may represent in amount ranging from 60 wt.% to 97 wt.%, preferably from 70 wt.% to 90 wt.%, more preferably from 75 wt.% to 90 wt.%, relative to the total weight of the composition.

### Oily Phase

The composition of the present invention comprises at least one oily phase, dispersed in the aqueous phase as described above.

The nature of the oily phase of the composition according to the present invention is not critical.

In particular, the oily phase comprises at least one oil.

The term oil refers to any fatty body in liquid form at room temperature (20-25° C.) and atmospheric pressure. These oils may be of animal, plant, mineral or synthetic origin.

The oils may be volatile or non-volatile.

The term “volatile oil” refers to any non-aqueous medium capable of evaporating from the skin or lips, in less than one hour, at room temperature (20-25° C.) and atmospheric pressure (760 mmHg). The volatile oil is a volatile cosmetic oil, liquid at room temperature. More specifically, a volatile oil has an evaporation rate of between 0.01 and 200 mg/cm²/min, inclusive.

The term “non-volatile oil” is intended to mean an oil remaining on keratin materials at ambient temperature and atmospheric pressure. More specifically, a non-volatile oil has an evaporation rate strictly below 0.01 mg/cm²/min.

To measure this evaporation rate, 15 g of oil or a mixture of oils to be tested are introduced into a crystallizer, 7 cm in diameter, placed on a scale located in a large 0.3 m³ chamber temperature-controlled at a temperature of 25° C., and humidity-controlled with a relative humidity of 50%. The liquid is left to evaporate freely, without stirring, by providing ventilation with a fan (PAPST-MOTOREN, reference 8550 N, rotating at 2700 rpm) positioned vertically above the crystallizer containing the solvent, with the blades directed toward the crystallizer and at a distance of 20 cm from the base of the crystallizer. The mass of oil remaining in the crystallizer is measured at regular intervals. The evaporation rates are expressed in mg of oil evaporated per surface area unit (cm²) and per time unit (minute).

The oil that is suitable for the present invention are not limited, it may be hydrocarbon-based, silicone-based or fluorine-based.

According to the present invention, the term “silicone oil” refers to an oil including at least one silicon atom, and in particular at least on Si—O group.

The term “fluorine oil” refers to an oil including at least one fluorine atom.

The term “hydrocarbon oil” refers to an oil containing primarily hydrogen and carbon atoms.

The oils may optionally include oxygen, nitrogen, sulfur and/or phosphorus atoms, for example, in the form of hydroxyl or acid radicals.

The oily phase may present in amount ranging from 0.5 wt.% to 20 wt.%, preferably from 0.5 wt.% to 15 wt.% and more preferably from 1 wt.% to 10 wt.%, relative to the total weight of the composition.

### Additional Cosmetic Active Agent(s)

Depending on the final purpose, the anhydrous composition according to the present invention can comprise an additional cosmetic active agent(s).

As additional cosmetic active agents that may be used in the anhydrous composition of the present invention, examples that may be mentioned include enzymes; flavonoids; moisturizers; anti-inflammatory agents; vitamins (for example, niacinamide); depigmenting agents; α-hydroxy acids; retinoids; antibacterial active agents; tensioning agents; ceramides; essential oils; UV-screening agents (or sunscreens), and mixtures thereof; and any other active agent that is suitable for the final purpose of the anhydrous composition.

It is easy for the skilled in the art to adjust the amount of the additional cosmetic active agent based on the final use of the anhydrous composition according to the present invention.

### Adjuvants or Additives

The anhydrous composition according to the present invention may also contain conventional cosmetic adjuvants or additives, for instance fragrances, additional surfactant different from those mentioned above (such as stearic acid, polysorbate 20), additional thickener, preserving agents (for example, phenoxyethanol) and bactericides, opacifiers, softeners, buffers, electrolytes such as sodium chloride, or a pH regulator (for example citric acid or potassium hydroxide), and mixtures thereof.

Needless to say, the skilled in the art will take care to select the optional adjuvant(s) added to the anhydrous composition according to the present invention such that the advantageous properties intrinsically associated with the anhydrous composition according to the present invention are not, or are not substantially, adversely affected by the envisaged addition.

According to a preferred embodiment, the present invention relates to a composition for brightening or whitening keratin materials in the form of an emulsion, comprising an oily phase dispersed in an aqueous phase, and comprising, relative to the total weight of the composition:
i) from 3 wt.% to 6 wt.% of at least one white particles selected from boron nitride, titanium dioxide, mica and a mixture thereof;
ii) from 0.3 wt.% to 1 wt.% of at least one linear or branched and/or crosslinked terpolymer of

at least one monomer (1) chosen from styrenesulfonic acid, ethylsulfonic acid and 2-methyl-2-[(1-oxo-2-propenyl]amino]-1-propanesulfonic acid, in free or partially or totally salified form,
a nonionic monomer (2) chosen from N,N-dimethylacrylamide and 2-hydroxyethyl acrylate, and
at least one polyoxyethylenated alkyl acrylate monomer (3) of formula (I) below:

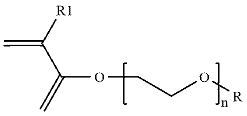

in which:
R1 represents a hydrogen atom,
R represents a linear or branched C8-C16 alkyl radical, and
n represents a number ranging from 1 to 10; and
   iii)an emulsifier system including:
      a) from 0.5 wt.% to 2 wt.% of potassium cetyl phosphate;
      b) from 0.5 wt.% to 2 wt.% of at least one polyoxyethylene oxide C12-C24 fatty acid ester selected from PEG 75 stearate, PEG 100 stearate, PEG 75 oleate, PEG 100 oleate, PEG 75 palmitate and PEG 100 palmite, and a mixture thereof; and
      c) from 0.5 wt.% to 2 wt.% of at least one glyceryl C12-C24 fatty acid ester selected from glyceryl laurate, glyceryl oleate, glyceryl stearate, glyceryl palmitate, and a mixture thereof.

### Method and Use

The composition according to the present invention is intended for topical application and can especially constitute a composition intended for brightening or whitening keratin materials, and especially human skin.

Thus, according to the second aspect, the present invention provides a cosmetic process for brightening or whitening keratin materials, in particular human skin, comprising applying the composition as defined above to the keratin materials.

The composition according to the present invention can have a suitable viscosity so that the consumer can apply it on keratin materials conveniently.

Advantageously, the composition has a viscosity of from 10 UD to 50 UD, preferably from 15 UD to 40 UD, more preferably from 18 UD to 30 UD, as measured with a viscometer RHEOMAT RM 180 with Mobile 3.

The viscosity is generally measured at 25° C. with a viscometer RHEOMAT RM 180 from the company Prorheo with Mobile 3 adapted to the viscosity of the composition to be tested (Mobile is chosen for having a measure between 10 and 90 for UD, Unit Deviation), the measure being made after 10 minutes rotating the mobile inside the composition at 200 rpm.

The composition according to the present invention can be used alone, or in combination with other cosmetic composition(s).

For example, the composition of the present invention can be used in combination with one or two other cosmetic compositions.

In one embodiment, the composition of the present invention and one or two other cosmetic compositions are contained in a two-pump or three-pump container, all cosmetic compositions have similar viscosity so that they can be pumped out in the same amount to be applied after being mixed by the consumer.

The present invention is illustrated in greater detail by the examples described below, which are given as non-limiting illustrations.

The percentages are weight percentages by active ingredient, or active matters.

In the examples that follow, the weight percentages are indicated relative to the total weight of the composition.

### Examples

### Example 1: Preparation of Serums

The serums according to invention formulas (inv.) 1-2 and comparative formulas (comp.) 1-3 comprising the ingredients shown in the following table were prepared, with all amounts expressed by percentages by weight of active matter with regard to the total weight of each serum.

| INCI NAME | Inv. 1 | Inv.2 | Comp. 1 | Comp. 2 | Comp. 3 |
|---|---|---|---|---|---|
| | Wt.% | Wt.% | Wt.% | Wt.% | Wt.% |
| XANTHAN GUM(KELTROL® CG-T from CP KELCO) | 0.20 | | | | |
| GLYCERYL STEARATE(ARLACEL™ 165 from CRODA) | 1 | 1 | 2 | 1 | 1 |
| PEG-100 STEARATE(ARLACEL™ 165 from CRODA) | 1 | 1 | 1 | | 1 |
| CETYL ALCOHOL | 0.28 | | | | |
| POLYACRYLATE CROSSPOLYMER-6(SEPIMAX™ ZEN from SEPPIC) | 0.6 | 0.6 | 0.6 | 0.6 | |
| ACRYLATES/C10-30 ALKYL ACRYLATE CROSSPOLYMER | | | | | 0.6 |
| WATER | QS100 | QS100 | QS100 | QS100 | QS100 |
| BORON NITRIDE | 2.4 | 2.4 | 2.4 | 2.4 | 2.4 |
| POLYSORBATE 20(TWEEN 20 from CRODA) | 0.2 | 0.2 | 0.2 | 0.2 | 0.2 |
| ISOPROPYL LAUROYL SARCOSINATE(ELDEW® from AJINOMOTO) | 2 | 2 | 2 | 2 | 2 |
| PENTYLENE GLYCOL | 2.5 | 2.5 | 2.5 | 2.5 | 2.5 |
| MICA | 1.1 | 1.1 | 1.1 | 1.1 | 1.1 |
| TITANIUM DIOXIDE | 1.8 | 1.8 | 1.8 | 1.8 | 1.8 |
| TIN OXIDE | 0.07 | | | | |
| BUTYLENE GLYCOL | 2 | 2 | 2 | 2 | 2 |
| CAPRYLYL GLYCOL | 0.3 | 0.3 | 0.3 | 0.3 | 0.3 |
| PHENOXYETHANOL | 0.6 | 0.6 | 0.6 | 0.6 | 0.6 |
| POTASSIUM CETYL PHOSPHATE(AMPHISOL® K from DSM) | 1 | 1 | | 1 | 1 |
| ETHANOL | 5 | 5 | 5 | 5 | 5 |

Serum of comparative formula 1 does not comprise any alkyl phosphate salt.

Serum of comparative formula 2 does not comprise any polyoxyethylene oxide C12-C24 fatty acid ester.

Serum of comparative formula 3 does not comprise any anionic terpolymer.

### Preparation Process

Serums listed above were prepared as follows:
1. preparing water phase: adding hydrotrope ingredient (PENTYLENE GLYCOL, BUTYLENE GLYCOL, CAPRYLYL GLYCOL, PHENOXYETHANOL, POTASSIUM CETYL PHOSPHATE), heating at 75° C., stirring at 500 rpm till complete dissolution;
2. adding hydrophilic thickening polymer (XANTHAN GUM, POLYACRYLATE CROSSPOLYMER-6 and/or ACRYLATES/C10-30 ALKYL ACRYLATE CROSSPOLYMER) into water phase to obtain a gelled aqueous phase;
3. preparing oil phsae: adding oil ingredients(GLYCERYL STEARATE, PEG-100 STEARATE, CETYL ALCOHOL, ISOPROPYL LAUROYL SARCOSINATE), heating to 75° C. until dissolved;
4. adding oil phase to gel phase with fast stirring at 1000 rpm for 10 minutes;
5. preparing a mixture of tween-20 (POLYSORBATE 20) and BN in water: adding BN and Tween 20 into small amount of water, then stirring in low speed to obtain a uniform suspension;
6. adding the mixture of tween-20 and BN in water, mix well at RT;
7. adding other charge (TITANIUM DIOXIDE, TIN OXIDE) and pearls (MICA).
8. adding alcohol (ETHANOL).

### Example 2: Evaluation of Serums

The stability and the viscosity of each composition were evaluated as followed.

Evaluation of stability

The stability was evaluated as follows.
1. Lumisizer stability: each serum was observed after storage for 16 hours at room temperature (25° C.) in the lumisizer rotating at 4000 rpm.
2. Standard stability: each serum was observed after storage at 45° C., 37° C., room temperature (25° C.) and 4° C. for 8 weeks.
3. Transportation stability: each serum was shaken in two directions, 1 hour for each direction.

A serum is stable if no oil out and/or white particles sediment is observed.

### Evaluation of Viscosity

The viscosity of each serum was determined at room temperature (25° C.) with a viscometer RHEOMAT RM 180 with Mobile 3 rotating at 200 rpm.

The stability and the viscosity of each serum were summarized in the following table.

| | | Inv. 1 | Inv.2 | Comp.1 | Comp.2 | Comp.3 |
|---|---|---|---|---|---|---|
| Standard stability | 45° C. | Stable | Stable | Stable | Stable | Stable |
| | 37° C. | Stable | Stable | Stable | Stable | Stable |
| | 25° C. | Stable | Stable | Stable | Stable | Stable |
| | 4° C. | Stable | Stable | Stable | Stable | Stable |
| Lumisizer stability | | Stable | Stable | unstable | unstable | unstable |
| Transportion stability | | Stable | Stable | unstable | unstable | unstable |
| Viscosity | | 25 | 20 | 20 | 6 | 13 |

It can be seen that the serums according to the present invention can comprise high content of white particles, be stable and have suitable viscosities so that the consumers can apply them conveniently, which was confirmed by 10 volunteers.

1. A composition for brightening or whitening keratin materials in the form of an emulsion, comprising an oily phase dispersed in an aqueous phase, and comprising:
i) from 2.0 wt. % to 10.0 wt. %of at least one white particles, relative the total weight of the composition;
ii) at least one anionic terpolymer as a thickener; and
iii) an emulsifier system including:
a) at least one alkyl phosphate salt;
b) at least one polyoxyethylene oxide C12-C24 fatty acid ester; and
c) at least one glyceryl C12-C24 fatty acid ester.

2. Composition of claim 1, wherein the white particles are selected from boron nitride, titanium dioxide, silica, pearl, mica, synthetic mica, mica based pearl pigment particles, composite particles comprising titanium dioxide and a substrate selected from alumina, silica, barium sulfate, glass, mica and synthetic mica, or mixtures thereof.

3. Composition of claim 1, wherein the white particles are present in the composition in an amount ranging from 2.5 wt. % to 8.0 wt. %, preferably from 3 wt. % to 6 wt. %, relative to the total weight of the composition.

4. Composition of claim 1, wherein the anionic terpolymer is selected from linear or branched and/or crosslinked terpolymers of at least one monomer (1) bearing an acid function being in free or partially or totally salified form with a nonionic monomer (2) chosen from N, N-dimethylacrylamide and 2-hydroxyethyl acrylate and at least one polyoxyethylenated alkyl acrylate monomer (3) of formula (I): 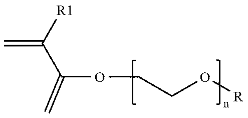
wherein:
R1 represents a hydrogen atom,
R represents a linear or branched C8-C16 alkyl radical, and
n represents a number ranging from 1 to 10.

5. Composition of claim 1, wherein the anionic terpolymer is present in an amount ranging from 0.1 wt. %to 5 wt. % relative to the total weight of the composition.

6. Composition of claim 1, wherein the alkyl phosphate salt is selected from monoalkyl phosphate salts of formula (II) : 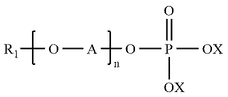
wherein:
R1 represents a linear or branched, alkyl or alkylene comprising from 4 to 24 carbon atoms, ;
A represents an alkylene group comprising from 2 to 10 carbon atoms;
n represents an integer ranging from 0 to 20;
X represents alkali metals .

7. Composition according to claim 1, wherein the alkyl phosphate salt is present in the composition in an amount ranging from 0.1 wt. %to 10 wt. % based on the total weight of the composition.

8. Composition according to claim 1, wherein the polyoxyethylene oxide portion of the C12-C24 fatty acid ester is a polyethylene glycol (PEG) having 50 to 150 units of PEG.

9. Composition according to claim 1, wherein the polyoxyethylene oxide C12-C24 fatty acid ester is present in the composition in an amount ranging from 0.1 wt. %to 10 wt. % based on the total weight of the composition.

10. Composition according to claim 1 , wherein the glyceryl C12-C24 fatty acid ester is selected from glyceryl laurate, glyceryl oleate, glyceryl stearate and glyceryl palmitate.

11. The composition according to claim 1 , wherein the glyceryl C12-C24 fatty acid ester is present in the composition in an amount ranging from 0.1 wt. %to 10 wt. % based on the total weight of the composition.

12. The composition according to claim 1 , wherein the weight ratio R of alkyl phosphate salt, polyoxyethylene oxide C12-C24 fatty acid ester and glyceryl C12-C24 fatty acid ester as defined below is 1: 0.5-2: 0.5-2, :
R=alkyl phosphate salt/polyoxyethylene oxide C12-C24 fatty acid ester/glyceryl C12-C24 fatty acid ester.

13. The composition according to claim 1 comprising, relative to the total weight of the composition:
i) from 3 wt. %to 6 wt. %of at least one white particle selected from the group consisting of boron nitride, titanium dioxide, mica and a mixture thereof;
ii) from 0.3 wt. %to 1 wt. %of at least one linear or branched and/or crosslinked terpolymer of
at least one monomer (1) selected from the group consisting of styrenesulfonic acid, ethylsulfonic acid and 2-methyl-2- [(1-oxo-2-propenyl] amino] -1-propanesulfonic acid, in free or partially or totally salified form,
a nonionic monomer (2) selected from the group consisting of N, N-dimethylacrylamide and 2-hydroxyethyl acrylate, and
at least one polyoxyethylenated alkyl acrylate monomer (3) of formula (I): 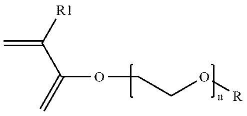
wherein:
R1 represents a hydrogen atom,
R represents a linear or branched C8-C16 alkyl radical, and
n represents a number ranging from 1 to 10; and
iii) an emulsifier system including:
a) from 0.5 wt. %to 2 wt. %of potassium cetyl phosphate;
b) from 0.5 wt. %to 2 wt. %of at least one polyoxyethylene oxide C12-C24 fatty acid ester selected from PEG 75 stearate, PEG 100 stearate, PEG 75 oleate, PEG 100 oleate, PEG 75 palmitate and PEG 100 palmite, and a mixture thereof; and
c) from 0.5 wt. %to 2 wt. %of at least one glyceryl C12-C24 fatty acid ester selected from glyceryl laurate, glyceryl oleate, glyceryl stearate, glyceryl palmitate, and a mixture thereof.

14. A cosmetic process for brightening or whitening keratin materials, comprising applying the composition of claim 1 on the keratin materials.
